(19) United States
(12) Patent Application Publication
(10) Pub. No.: US 2002/0177767 A1
(43) Pub. Date: Nov. 28, 2002

(54) Sensor for biopotential measurements

(76) Inventors: Steve Burton, Midlothian, VA (US); Mark J. Licata, Doswell, VA (US); James Mitchell, Midlothian, VA (US)

Correspondence Address: JOHN H. THOMAS, P.C., 1561 EAST MAIN STREET, RICHMOND, VA, 23219, US

(21) Appl. No.: 10/121,541

(22) Filed: Apr. 12, 2002

Related U.S. Application Data
(63) Continuation-in-part of application No. 09/773,921, filed on Feb. 2, 2001.
(60) Provisional application No. 60/204,603, filed on May 16, 2000.

(51) Int. Cl. A61B 5/04

(52) U.S. Cl. 600/397; 607/153

(57) ABSTRACT

A sensor for biopotential measurements is designed to detect low voltage electrical signals on a subject's skin surface. A plurality of soft elastomeric bristles are arranged about the surface of the skin. Various bristles contain a wick, made of polyolefin, polyester or nylon, extending along its center axis with one end protruding from the bristle and another end in contact with a fluid reservoir. The wick is saturated with an electrically conductive liquid, such as a salt solution. The solution may contain a surfactant. The rheological properties of the electrically conductive liquid are optimized for predictable flow through the wick onto the skin surface. An electrode is positioned in the vicinity of the wick and the reservoir. Alternatively, a sensor comprises a plurality of hollow, soft elastomeric bristles filled with a hydrogel. An electrically conductive cap provides the electrical contact between the hydrogel and the electrical circuit.

### CROSS-REFERENCE TO RELATED APPLICATION

The present application is a continuation-in-part of U.S. patent application Ser. No. 09/773,921, filed Feb. 2, 2001, which claims the benefit of priority to provisional patent application Serial No. 60/204,603 to Mark Licata and James Mitchell, filed on May 16, 2000, entitled “Electrode For Biopotential Measurements”, both of which are hereby incorporated by reference.

### TECHNICAL FIELD

This invention relates generally to the field of sensors for measuring electrical potentials obtained from the surface of the skin including, for example, electroencephalogram (EEG), electrocardiogram (ECG), or electromyogram (EMG) sensors.

### BACKGROUND OF THE INVENTION

In the past, electroencephalogram (EEG), electrocardiogram (ECG), and electromyogram (EMG) electrodes have needed the assistance of technicians for proper use, and thus have been relegated for use in clinical environments. With the advent of new modern electronic devices, there has developed a need for an electrode sensor that patients may use at home. These new devices allow patients to use new portable medical devices that require electrodes. The electrode needs to be non interfering with the patients hair and needs to be designed so that its use does not require chemicals or gels that can leave a mess. The prior art does not satisfy these requirements.

U.S. Pat. No. 3,508,541, entitled “Electrode Construction” to R. M. Westbrook et al. discloses an electrode device comprising an electrode element formed of an intimately bonded homogeneous mixture of finely divided Ag and AgCl. An elongated resilient skin engaging member, such as a disposable hollow sponge, holds an electrolyte, such as a sodium chloride gel. Additionally, Westbrook et al. discloses an electrode device which is simply applied to the scalp, eliminates motion artifacts, and regardless of such factors as hair tonics, sunburn, hair length/thickness, or perspiration obtains a good, low impedance, contact. The electrode of Westbrook et al. makes no suggestion that a plurality of the elongated resilient skin engaging members would be beneficial in achieving improved contact, and the electrode device configuration is complicated and would be expensive to mass produce.

U.S. Pat. No. 4,195,626 to Schweizer entitled “Device for the Production and Application of Body Stimuli Devices”, discloses a biofeedback chamber for applying stimuli and for measuring and analyzing a subject's reaction to control the stimuli. One of the stimulus applicators is a flexible laminar electrode comprising a plurality of reinforced filament bundles, a hollow reservoir and a porous reservoir for holding an electrolyte, and a metal conductor embedded in the porous reservoir. The filament bundles provide capillary action to deliver electrolyte from the porous reservoir to a patient's skin. Besides the fact that Schweizer's disclosure is directed to an electrode for a stimulus applicator as opposed to an electrode for measuring biopotentials, Schweizer teaches away from the present invention in that a flexible laminar electrode is formed of a flexible support, two plastic sheets, yet the filament bundles are stiffened with a reinforcement jacket.

U.S. Pat. No. 4,967,038 to Gevins et al. entitled “Dry Electrode Brain wave Recording System”, discloses a semi-rigid helmet containing a plurality of rubber multi-contact electrodes. The electrodes comprise a gold-plated metal pin with one end formed in a rubber base. A plurality of pyramid-shaped rubber fingers, extending from the base, are terminated with conductive round metal tips. Metal flexible wire, attached at a solder point to the pin within the base, extends through the center of each finger to their tips. The flexibility of the multiple fingers allows the electrode to adapt to the local contours of a head. Having redundant, multiple contact points with the scalp improves the connection since it is not dependent on the impedance at a single small point. The rubber multi-contact electrodes of Gevins et al. do not incorporate a mechanism for applying an electrolyte to the scalp in order to improve electrical contact, improve comfort by moistening the skin, and reducing the electrical resistance of the skin. Additionally, Gevens et al. requires electrical conductivity in each of the fingers of their electrode.

U.S. Pat. No. 5,211,184 to Yee et al., entitled “Method and Apparatus For Acupuncture Treatment”, discloses an electrode assembly for applying an electrical signal to the skin surface. The electrode assembly comprises a hollow body filled with an electrically conductive fluid, a wick-like material for delivering the fluid to a point where one end of the material is in contact with the skin surface, and a metallic cap attached to a second end of the material. Besides the fact that the Yee et al. disclosure is directed to an electrode for applying an electrical signal as opposed to an electrode for measuring biopotentials, there is no suggestion that a plurality of wicks extending from the hollow body would be beneficial in achieving improved contact with the skin surface.

U.S. Pat. No. 6,067,464 to Musha, entitled “Electrode”, discloses an electrode for measuring bio-electric waves. The electrode comprises a support member, a piece of absorbent fiber, and a non-corrosive lead. The support member, made of an insulating material such as ceramic, plastic or heat treated synthetic fibers or felt, is disk-shaped with a hollow, concentric cylindrical projection. The absorbent fiber, made of felt, cotton or synthetic fibers, is mounted in the projection on the support with one end extending beyond the edge of the projection. Alternatively, the absorbent fiber may comprise a bundle of carbon powder impregnated hard felt rods with rounded tips. Electrically conductive fluid, such as saline solution, is introduced into the support through an insertion hole formed opposite the projection, and is absorbed by the absorbent fiber. The electrically conductive fluid may also comprise various skin conditioners, counterirritant materials, anti-inflammatory agents, and astringents. A lead, made of a bundle of carbon fibers, makes contact with the absorbent fiber through the wall of the projection. Musha teaches away from the present invention by incorporating an insertion hole for introducing electrically conductive fluid into the electrode before and during use as opposed to including a reservoir for holding sufficient electrically conductive fluid for the life of the electrode. Additionally, there is no suggestion that a support comprising a plurality of projections, each with an absorbent fiber, would be beneficial in achieving improved contact with the skin surface.

These conventional sensor configurations described above each fail to disclose at least a single significant attribute of the present invention. What is needed is an electrode which may be used on open skin, or skin covered with hair, does not require the use of external gels or waxes to obtain adequate electrical conduction to the skin surface, may be comfortably worn for long periods of time, and may be properly applied by an individual's scalp without the assistance of a technician.

### BRIEF SUMMARY OF THE INVENTION

One advantage of the invention is that it provides a sensor which can be used on open skin, or skin covered with hair and does not require the use of external gels or waxes to obtain adequate electrical conduction to the skin surface.

Another advantage of the present invention is that it provides a sensor which can be comfortably worn for long periods of time.

Yet, another advantage of the present invention is that it provides a sensor which can be applied by the individual wearing the sensor. Hence, no technician is required.

To achieve the foregoing and other advantages, in accordance with all of the invention as embodied and broadly described herein, a sensor for biopotential measurements comprising at least one elastomeric bristle having a base and a tip with a channel running there between and a porous wick extending through the channel, the tip contacting a skin surface; a reservoir containing an electrically conductive material is formed at the base of said elastomeric bristle; and an electrode for detecting electrical potential. The porous wick transports the electrically conductive material from the reservoir to the elastomeric bristle tip in order to conduct an electrical signal obtained from the skin surface, moisten the skin surface, and reduce the electrical resistance of the skin surface.

In yet a further aspect of the invention, a sensor for biopotential measurements wherein the reservoir is formed of at least one of: a porous material; and a hollow vessel capable of holding an electrically conductive liquid. The rheological properties of the electrically conductive liquid may be optimized for predictable flow through the porous wick onto the skin surface.

In yet a further aspect of the invention, a sensor for biopotential measurements comprising: a plurality of physically linked and electrically isolated elastomeric bristles, each having a base and a tip with a channel running there between, the tip contacting a skin surface; and an electrode for detecting electrical potential. The channel may be filled with a hydrogel material which is formulated to have high electrical conductivity in order to conduct an electrical signal obtained from the skin surface, moisten the skin surface, and reduce the electrical resistance of the skin surface.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and form a part of the specification, illustrate embodiments of the present invention and, together with the description, serve to explain the principles of the invention.

FIG. 1 is a cross-sectional view of an individual elastomeric bristle according to an embodiment of the present invention.

FIGS. 2A and 2B are exterior and interior views, respectively, of a surface comprising a plurality of elastomeric bristles with wicks in accordance with an embodiment of the present invention.

FIG. 3 is a cross-sectional view of an individual elastomeric bristle according to an embodiment of the present invention.

FIG. 4 is a cross-sectional view of elastomeric bristles with an electrode cap according to an embodiment of the present invention.

FIG. 5 is a cross-sectional view of an individual elastomeric bristle showing an electrode embedded in the elastomeric bristle according to an embodiment of the present invention.

FIG. 6 is a cross-sectional view of an aspect of an embodiment of the present invention showing an electrode and electrode cap fastened to a sensor top.

FIG. 7 is an external view of a sensor according to an embodiment of the present invention.

FIG. 8 is a side elevation cross sectional view of an alternative embodiment of a sensor in accordance with the present invention.

FIG. 9 is a side elevation view of a sensor strip embodying the present invention.

FIG. 10 is a top plan view of the sensor strip shown in FIG. 9.

FIG. 11 is a bottom view of the sensor strip shown in FIG. 9.

FIG. 12 is a perspective view of a further embodiment of a sensor assembly in accordance with the present invention.

FIG. 13 is a side elevation view of a sensor as seen in FIG. 12.

FIG. 14 is a side elevation exploded view of a sensor as shown in FIGS. 12 and 13.

### DETAILED DESCRIPTION OF THE INVENTION

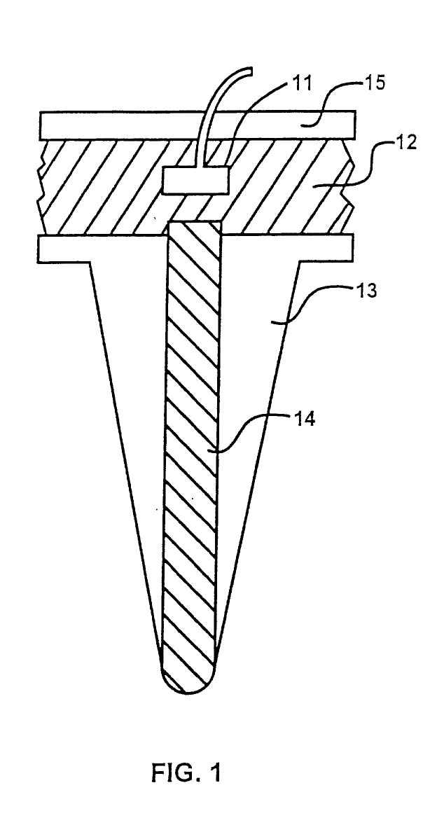

FIG. 1 is a cross-sectional view of an individual elastomeric bristle according to an embodiment of the present invention. As shown, a soft elastomeric bristle 13 contains a wick 14 of suitable material that extends through a channel in the center of the bristle 13. One end of the wick 14 protrudes from the end of the elastomeric bristle 13 to contact a skin surface. The other end of the wick 14 extends past the elastomeric bristle 13 into a fluid reservoir area 12. The fluid reservoir preferably has a sensor top 15 capping it. In the preferred embodiment, the wick material is polyolefin, but other materials are suitable including polyester or nylon.

The wick 14 may be saturated with an electrically conductive liquid, such as a solution of 0.2 to 1.0 molar sodium chloride, potassium chloride, sodium bicarbonate, or other salt solution. The solution serves to conduct the electrical signal obtained from the skin surface to an electrode 11 in the fluid reservoir area 12. The solution may also serve to moisten the skin surface and reduce the electrical resistance of the skin. The solution may also contain a surfactant to facilitate skin moistening, for example, 5 g/liter of sorbitan laurate.

The fluid reservoir 12 may be composed of a porous material capable of holding sufficient solution for the life of the sensor. Alternatively, the fluid reservoir 12 may be a hollow vessel to contain a volume of electrically conductive solution. The wick 14 conducts the solution to the skin surface as the fluid reservoir 12 is gradually depleted. When the fluid reservoir is fully depleted, it may be refilled by a variety of methods including reverse capillary action.

The rheological characteristics of the electrically conductive liquid may be manipulated by selecting specified components to form the electrically conductive liquid's composition. Particular materials may be mixed to create a solution of electrically conductive liquid with a specific viscosity. Additionally, various wick materials may exhibit different capillarity. In constructing the present invention, the composition of the electrically conductive liquid and the wick material may be predetermined for optimum control of the flow rate of the electrically conductive liquid through the wick 14. Flow control preferably determines the amount of skin surface wetting. Optimization of the rate of capillary action and viscosity may be performed to compensate for common chemical products applied to the hair and scalp, such as tonics, dyes, sprays and gels, which may react with the components of the sensor.

Alternatively, the fluid reservoir 12 may also be a volume of porous material loaded with a solution that is in fluid contact with the wick 14. The material may be of such suitable material as cellulose acetate or urethane foam.

At the bottom of the fluid reservoir 12, or at the junction of the wick 24 and porous reservoir material, an electrode 11 may be placed to detect the electrical potential conducted through the wick 14. The electrode 11 may be connected to instrumentation capable of amplifying and processing the electrical signal. The electrode 11 may be composed of any material capable of ionic transduction, such as a combination of silver and silver chloride.

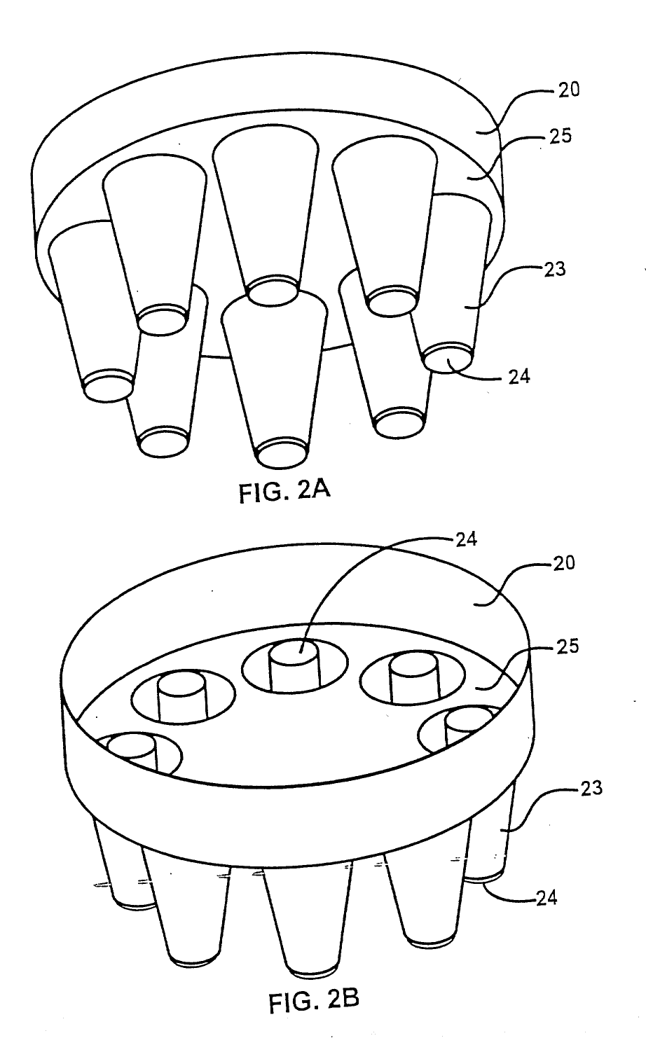

FIGS. 2A and 2B are exterior and interior views, respectively, of a surface comprising a plurality of elastomeric bristles with wicks in accordance with an embodiment of the present invention. As illustrated, a plurality of elastomeric bristles 23 may be physically linked to form a comb 25. The comb 25 is preferably made of a stiff but flexible material such as molded silicon rubber. Each of the elastomeric bristles 23 contains a wick 24 at its core. Each wick 24 may be coupled to a fluid reservoir 12 bound by an outer wall 20. The electrical signals obtained from the elastomeric bristles 23 may be summed in the fluid reservoir 12.

Experimentation has determined that it is not required that every elastomeric bristle 23 on the comb 25 be electrically conductive. In order to achieve a good measurement of biopotential and provide a sensor that is comfortable and securely applied to a skin surface, yet reduce complexity of the device and cost of manufacturing, the comb 25 may be formed with several of the elastomeric bristles 23 as “dummy” bristles that do not provide any electrical conductivity.

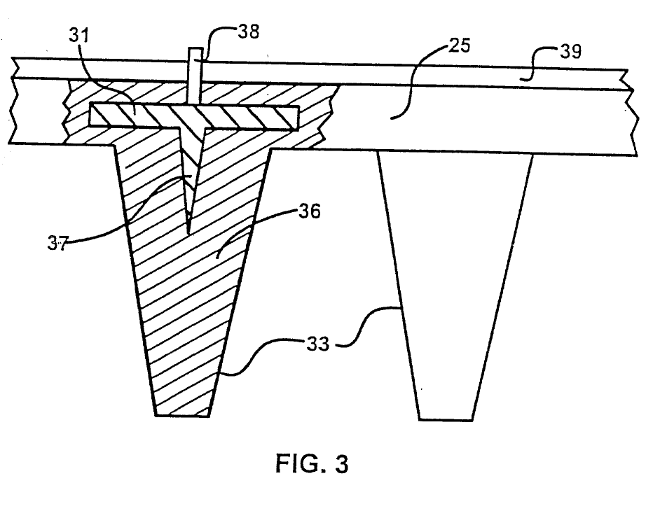

FIG. 3 is a cross-sectional view of an individual elastomeric bristle according to an embodiment of the present invention. Electrode 31 may be formed such that a large surface area is exposed to the fluid reservoir in order to conduct a strong electrical signal from the bristle 33. The surface area may take the form of a disk. As illustrated, the electrode structure may have conductive spikes 37 positioned to align coaxially with each of the elastomeric bristles 33. One skilled in the art will recognize that many different shapes, for example, a cylinder may be used for the conductive spikes 37. The electrode 37 may provide for a connector 38 to protrude from one side of the disk-shaped electrode 37 and extend externally from the sensor top 39 in order to facilitate connection with external circuitry and a sensor mounting structure.

Alternatively, the material of a porous fluid reservoir may be manufactured in such a way as to have the requisite electrical conductivity as a separate electrode. Preferably, the porous fluid reservoir material may be coated with a combination of silver and silver chloride particles. An electrical connection may then be made between the reservoir material and the measuring instrumentation.

Preferably, the elastomeric bristles 33 are of such a stiffness, or durometer, as to provide for isolation of the sensor from mechanical shock. The end of the elastomeric bristles 33 in contact with a skin surface may remain stationary as the body of the sensor, and the device to which it is coupled, have a certain degree of freedom of movement.

Each elastomeric bristle 33 contains a core 36 of conductive hydrogel that extends through the center of the bristle. One end of the hydrogel core protrudes from the end of the elastomeric bristle 33 to contact the skin surface. The other end of the hydrogel core 36 is in contact with an electrode 37.

The hydrogel material is preferably formulated to have high electrical conductivity. The hydrogel serves to conduct the electrical signal obtained from the skin surface to the electrode 37. The hydrogel may also serve as a source of moisture to reduce the electrical resistance of the skin surface. The hydrogel may contain a surfactant to facilitate skin moistening.

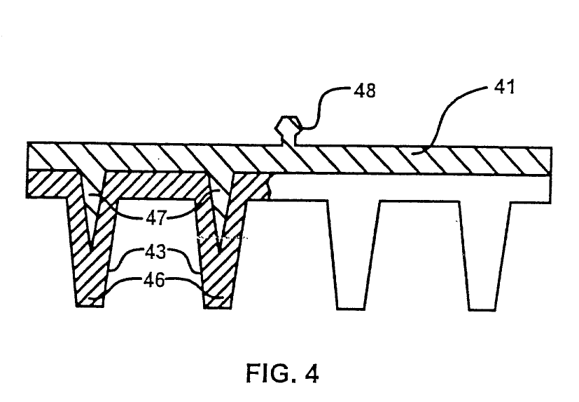

FIG. 4 is cross-sectional views of elastomeric bristles with an electrode cap according to an embodiment of the present invention. As with the first embodiment, a plurality of elastomeric bristles 43 may be physically linked to form a comb structure. The electrical signals may be obtained from each individual elastomeric bristle 43 and are summed at electrode 41. As shown, the electrode 41 is also the reservoir top.

The electrode 41 may be connected to instrumentation capable of amplifying and processing the electrical signal. The electrode 41 can be composed of any electrically conductive material, for example, a combination of silver and silver chloride.

The electrode 41 may be formed such that a large surface area is exposed to the core 46 of each of the elastomeric bristles 43 in order to conduct a strong electrical signal from the hydrogel. The surface area may take the form of a disk. Additionally, the electrode 41 provides for a connector 48 to protrude from one side of the disk-shaped electrode 41 and extend externally from the sensor in order to facilitate connection with external circuitry and a sensor mounting structure. In a modified electrode structure, conductive spikes 47 may be formed on the face of the disk opposite the connector 48. The conductive spikes 47 may be positioned to align coaxially with each of the elastomeric bristles 43.

Preferably, the elastomeric bristles 43 are of such a stiffness, or durometer, as to provide for isolation of the sensor from mechanical shock. The end of the hydrogel cores 46, in contact with the skin surface, can remain stationary as the body of the sensor, and the device to which it is coupled, have a certain degree of movement.

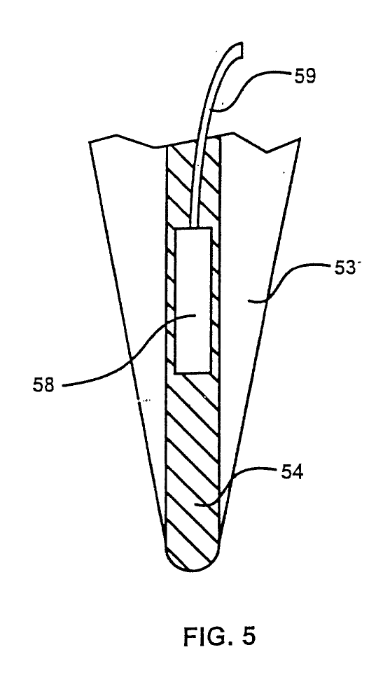

FIG. 5 is a cross-sectional view of an individual elastomeric bristle 53 showing an electrode 58 embedded in the elastomeric bristle 53 according to an embodiment of the present invention. In this embodiment, the conductive core 54 of the elastomeric bristle 53 may include any conductive material such as a wick or hydrogel. An electrode lead 59 may be used to conduct the signal out of the sensor assembly.

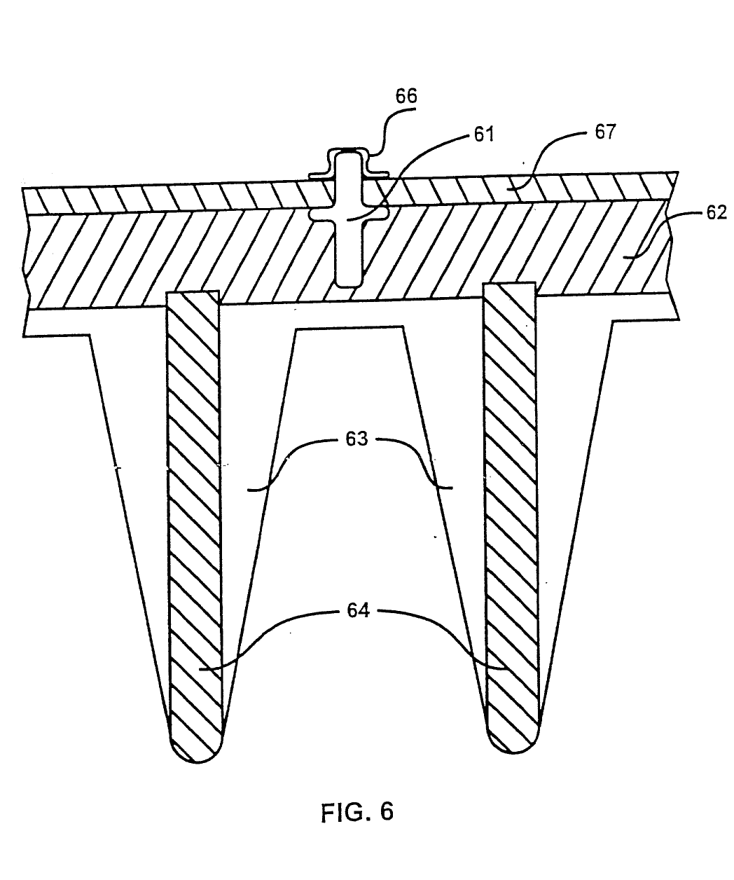

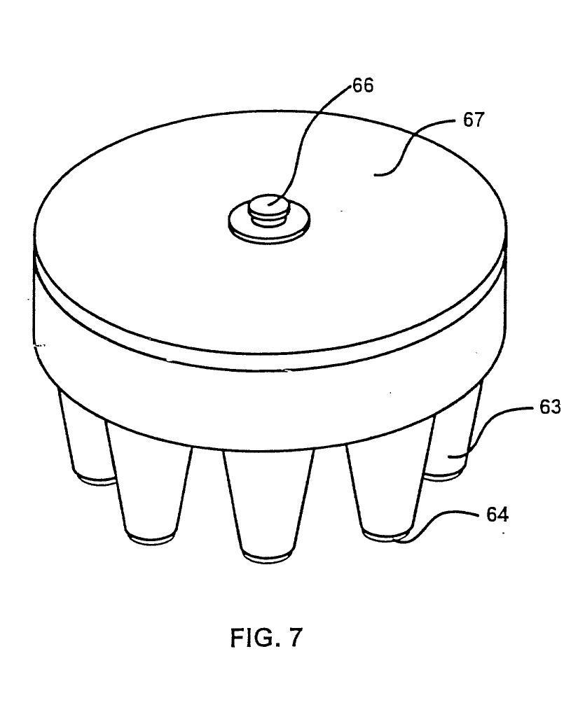

FIG. 6 is a cross-sectional view of an aspect of an embodiment of the present invention showing an electrode 61 and electrode cap 66 fastened to a sensor top 67. In this embodiment, the biopotential signals are conducted up the conductive cores 64 from each of the bristles 63 and are preferably summed in the reservoir 62. FIG. 7 is an external view of a sensor according to the embodiment illustrated in FIG. 6.

For any of the disclosed embodiments of the present invention, the sensor assembly may be disposable like a pen or an ink cartridge for a printer. This allows change over for different users or replacement.

The embodiments of the present invention described thus far herein discuss the use of a bristle. The wick of the present invention, however, does not require the use of a bristle. For instance, a sensor that is positioned directly on a section of skin simply requires a membrane wick. A membrane, like a bristle, has the controlled porosity that allows the predictable flow of the fluid from a reservoir to the skin surface.

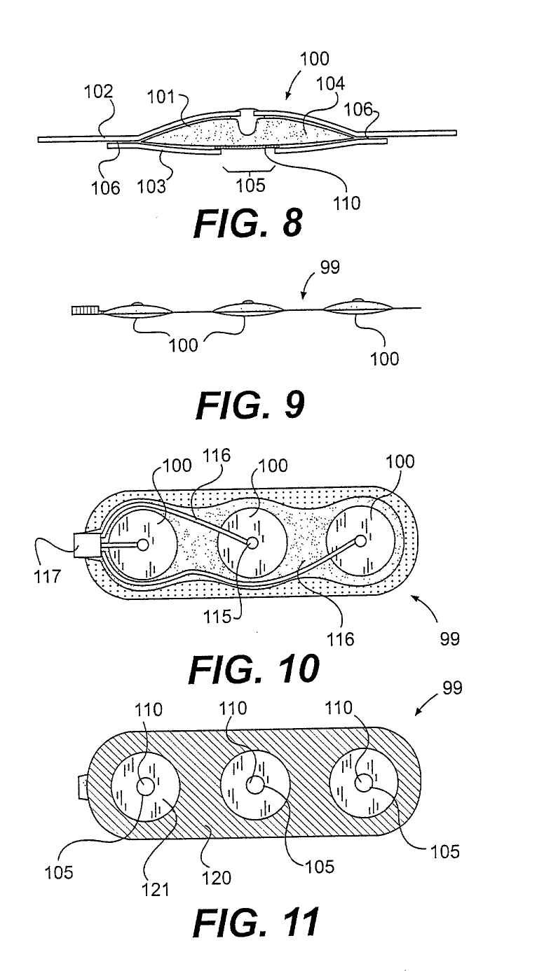

FIGS. 9 through 11 illustrate a sensor strip 99 that is comprised of three separate sensors 100. FIGS. 8 through 11 illustrate the detail of the sensor strip 99 and each of the sensors 100. Each sensor 100 is made from two nonporous films 102 and 103 that define the outside of a reservoir 101. The bottom film 103 further has a round aperture 105. The top film 102 and bottom film 103 are sealed together around the perimeter of the reservoir 101 along edge 106. Typically, the films 102 and 103 are heat sealed along perimeter 106. However, adhesives or cohesives or other methods of joining the film may be used to form the sealed reservoir 101. The aperture 105 is covered by a wicking membrane 110. The wicking membrane 110 is sealed around the aperture 105 to the film 103. Also, electrode 115 extends downwardly into the reservoir 101 and also through film 102 so that it is accessible outside of the reservoir.

The reservoir 101 as shown contains a hydrophilic foam 104. The hydrophilic foam 104 is saturated with an electrically conductive material. The electrically conductive material is allowed to wick out of the reservoir 101 through the wicking membrane 110. In operation, therefore, the sensor 100, when applied to a patient's skin, moistens the patients skin and allows for a fluid communication between the skin and the electrode 115.

As shown in FIG. 10, the electrodes 115 come into contact with electrical traces 116 which are likewise connected to the electrical connector 117. On the bottom of the sensor strip 99, there is shown a pattern of adhesive 120 which defines areas 121 that are moistened with the electrically conductive fluid that wicks through the aperture 105 through the wicking membrane 110. The adhesive 120 acts to seal off each area 121 so that there is no disruption or damage to the signal obtained by each of the individual sensors 100. In other words, the adhesive 120 helps prevent bridging of the signals between the sensors 100.

In one preferred embodiment, the exposed area 21 that is intended to be moistened with the electrically conducted fluid has a 10 mm circular diameter. (An area of approximately 0.78 cm²) The controlled rate of flow through the wicking membrane 110 is 1.3 microliters per minute per cm². Assuming that this preferred sensor 100 having the above-referenced dimensions were to be used for twelve hours, it would be necessary, therefore, to have at least one ml of electrically conductive material in the reservoir 101 in order to maintain sufficient moisture and contact through the entire time period.

The microporous wicking membrane 110 may be made from any type of material which allows for a controlled rate of directional flow of fluid out of a reservoir such as reservoir 101. The wick may comprise fibers as described in the earlier embodiments of the bristle detailed herein. The wick may be a porous membrane that is perforated mechanically or chemically. Commercially available wicking membranes or similarly active films are available from Tredegar Industries sold under the VISPORE trademark. The specific film or membrane that may be appropriate for a given application will vary with the specific parameters of the application including but not limited to the following: the fluid that will flow through the wicking membrane, the desirable rate of flow through the wicking membrane, the size of the reservoir, the size of the aperture between the reservoir and a patient's skin (once it is applied), the desired useful life of the sensor, etc. Trial and error or prior experience may be necessary to accurately select a specific wicking membrane that would be appropriate for a specific application.

The electrically conductive material is preferably a salt solution that is adapted to pick up the ionic current signals given off by a patient's skin. This solution is typically an aqueous solution of water and sodium chloride or potassium chloride, although other salts such as sodium bicarbonate may also be used. This solution will also typically include a small amount of preservative such as EDTA or methyparaben. There may also be included flow control agents or surfactants such as carboxymethylcellulose or polyethylene glycol. In a preferred embodiment of a sensor having a twelve-hour useful life and a controlled flow rate of 1.3 microliters per minute per cm², one ml of electrically conductive material (salt solution) is required. The concentration of the salt solution is preferably 0.5 molar; however, any solution within the range of 0.2M to 1.0M could be acceptable depending on other of the sensor parameters and specifications.

Preferably, a hydrophilic foam such as foam 104 is used to hold the electrically conductive material within the reservoir 101. This foam assists in the control of the rate of flow of liquid out of the reservoir 101. Cellulose acetate may be used as a type of hydrophilic foam. Other types of medical foams including those sold by Rynel may also be acceptable. Again, the specific type of hydrophilic foam will depend on many of the same variables noted earlier in connection with the selection of a wicking membrane.

The electrode 115 is typically a silver/silver chloride transducer that converts ionic current to electric current. These silver/silver chloride transducers are conventionally available from Select Engineering. The electrode may be solid silver/silver chloride, or it may be plastic with a thin deposit of silver chloride on its outer layer. Also, the electrode could itself comprise merely an extension of the electrical trace 116 which is used to connect the signal from the inside of a sensor 100 to an electrical connector 117. Also, it is not necessary that the transducer necessarily be silver chloride. Other types of transducers that convert ionic to electric current may be used.

As shown in FIGS. 9 through 11, the sensor strip 99 has three sensors 100. This linear configuration, as well as the number of sensors 100, is a matter of convenience and design. Only one sensor is necessary to pick up a signal from the body (although a single sensor would require an additional ground lead attached to a patient). In the sensor strip 99 shown, the center sensor is the grounded sensor while the two sensors on either end detect the current signals created by the patient's body. And specifically in the sensor application of an EEG sensor, two electrodes are preferred in order to obtain a bipolar referential measurement. Still further, the sensor strip 99 as shown illustrates the three sensors 100 shown in a line. The specific geometry of those sensors is not limited to this design. Other configurations include, if desirable, more or less sensors that have different or variable geometries.

The film layers 102 and 103 may be made of any nonporous and flexible polymer. It is preferable that the film 102 especially be able to receive an electrical tracing such as electrical trace 116. Also, it is desirable that the films 102 and 103 be able to receive an adhesive such as adhesive 120 that allows for the sensor to be securely placed onto a patient, and yet also conform to the contours of the patient.

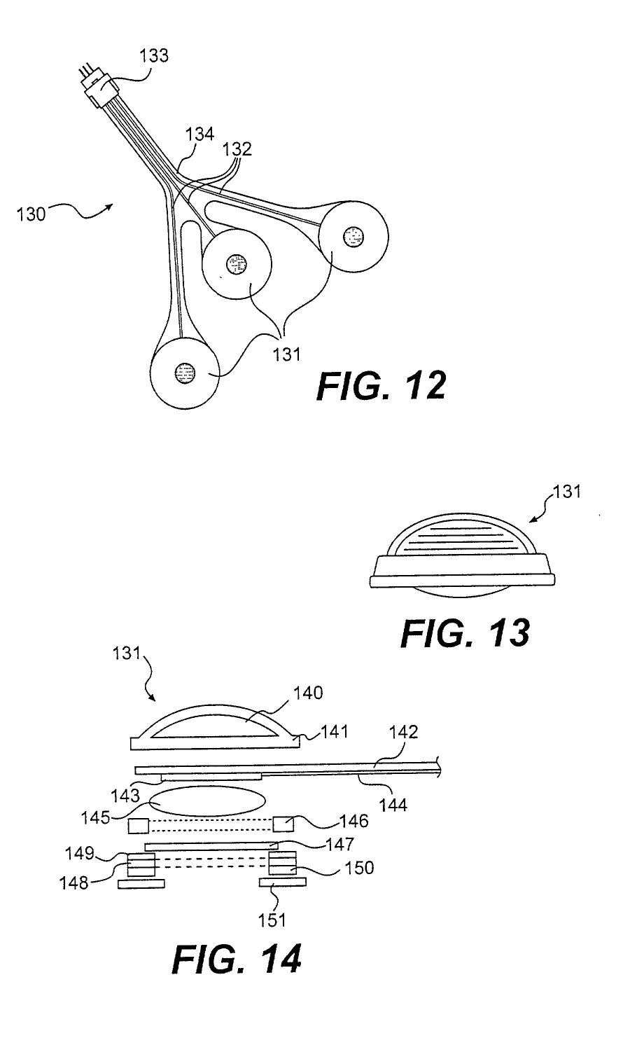

FIGS. 12 to 14 illustrate a still further embodiment of a sensor assembly. The sensor assembly 130 includes three sensors 131. Sensors 131 are electrically connected to connector 133 along conductive traces 132. The conductive traces 132 are layered onto a polyester film 134 which serves to connect the entire assembly 130. In a preferred embodiment, the distance from the connector 133 to the middle sensor 131 is 110 mm. The distance from the center sensor 131 to either of the side sensors is 60 mm. The angle formed by the two straight lines from the outside sensors to the central sensor is 132 degrees. The connector 133 is a conventional Molex® 3-prong connector.

Turning now to FIGS. 13 and 14, there is shown a single senor 131. The sensor 131 is made up of a pull grip/positioning tab 140 that is part of an injection molded plastic cap 141. The cap 141 is attached to a polyester film 142 by means of an adhesive. The film is a 4 mil polyester. A silver trace 144 is imprinted on the polyester film 142. The traces 144 are made of Ag500 Conductive Products ink. The trace 144 leads to an electrode 143 which is an area of silver/silver chloride that is imprinted on the film 142. The actual sensor electrode 143 is made of Conductive Products 50/50 Ag500-AgCl500 ink. A hydrophobic foam donut 146 is adhered to the film 142. The donut 146 defines a reservoir therein into which is placed hydrophylic foam 145. This foam is Rynel 562 medical urethane foam 19×6×3 mm. The sensors 131 may hold variable volumes of conductive material as discussed earlier herein. The membrane wick film 147 is mounted around the hole defined by the foam donut 146. The specific film is a Tredegar VISPORE 40 Hex PE. A further polyester film 148 is adhered to the foam donut 146 by a layer of adhesive 149. This film 148 secures the wick film 147 around the reservoir defined by the donut 146. A further layer of adhesive 150 is a means for adhering the sensor to the forehead of a patient. For storage and shipment, that adhesive 150 is covered with a wax contact paper 151.

Finally, it is preferred that the sensors described herein be effectively radiotranslucent. Of course, the electrode (preferably silver/silver chloride) and electrical trace (preferably silver) are radioopaque, but the majority of the sensor is plastic, foam, adhesive, etc. and transparent to x-ray. In this way, there is an obvious advantage in not having to remove a patient's sensor if an x-ray or other radiography is necessary.

The foregoing descriptions of the preferred embodiments of the present invention have been presented for purposes of illustration and description. They are not intended to be exhaustive or to limit the invention to the precise forms disclosed, and obviously many modifications and variations are possible in light of the above teaching. The illustrated embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A sensor for biopotential measurements comprising:
a porous wick adapted to contact a skin surface,
a reservoir containing an electrically conductive material adjacent to and in fluid contact with the wick, and
an electrode for detecting electrical potential,
wherein the wick transports the electrically conductive material from the reservoir through the wick at a predetermined, controlled rate of flow.

2. A sensor for biopotential measurements according to claim 1, wherein the reservoir further comprises a porous material.

3. A sensor for biopotential measurements according to claim 2, wherein the porous material is selected from the group consisting of:
cellulose acetate and urethane foam.

4. A sensor for biopotential measurements according to claim 1, wherein said electrically conductive liquid is a solution of 0.2 to 1.0 molar salt solution.

5. A sensor for biopotential measurements according to claim 4, wherein said salt solution is selected from the group consisting of:
sodium chloride, potassium chloride, and sodium bicarbonate.

6. A sensor for biopotential measurements according to claim 4, wherein the salt solution further comprises a surfactant.

7. A sensor for biopotential measurements according to claim 2, wherein the electrode is an electrically conductive coating on said porous material of the reservoir.

8. A sensor for biopotential measurements according to claim 7, wherein the electrically conductive coating comprises silver and silver chloride.

9. A sensor for biopotential measurements according to claim 1, wherein the porous wick is made of a material selected from the group consisting of:
polyolefin, polyester, and nylon.

10. A sensor for biopotential measurements according to claim 1, wherein the electrode is made of a composition comprising silver and silver chloride.

11. A sensor for biopotential measurements comprising:
a reservoir containing an electrically conductive material wherein the reservoir has an aperture,
a porous wicking membrane that is sealed around and covers the aperture, and
an electrode for detecting electrical potential,
wherein the wicking membrane transports the electrically conductive material from the reservoir through the wicking membrane at a predetermined, controlled rate of flow.

12. A sensor for biopotential measurements according to claim 11, wherein the reservoir further comprises a porous material.

13. A sensor for biopotential measurements according to claim 11, wherein the electrically conductive liquid is a salt solution.

14. A sensor for biopotential measurements according to claim 13, wherein the salt solution has a concentration of 0.2 to 1.0 molar.

15. A sensor for biopotential measurements according to claim 13, wherein the salt solution comprises one or more compounds selected from the group consisting of sodium chloride, potassium chloride and sodium bicarbonate.

16. A sensor for biopotential measurements according to claim 11, wherein the electrically conductive material comprises a surfactant.

17. A sensor for biopotential measurements according to claim 16, wherein the surfactant comprises sorbitan laurate.

18. A sensor for biopotential measurements according to claim 11, wherein the aperture has an area of approximately 0.78 cm².

19. A sensor for biopotential measurements according to claim 18, wherein the controlled rate of flow across the wicking membrane is approximately 1.3 μL/min./cm².

20. A sensor for biopotential measurements according to claim 11, wherein the reservoir contains approximately one ml of electrically conductive material.
